(19) United States
(12) Patent Application Publication
(10) Pub. No.: US 2026/0058023 A1
(43) Pub. Date: Feb. 26, 2026

(54) ESTIMATION DEVICE, ESTIMATION METHOD, AND RECORDING MEDIUM

(71) Applicant: NEC Corporation, Tokyo (JP)

(72) Inventors: Keisuke Suzuki, Tokyo (JP); Yuki Kosaka, Tokyo (JP); Kosuke Nishihara, Tokyo (JP); Kentaro Nakahara, Tokyo (JP); Fumiyuki Nihey, Tokyo (JP); Mana Hashimoto, Tokyo (JP)

(73) Assignee: NEC Corporation, Tokyo (JP)

(21) Appl. No.: 19/291,666

(22) Filed: Aug. 6, 2025

(30) Foreign Application Priority Data
Aug. 26, 2024 (JP) 2024-144416

(51) Int. Cl. G16H 50/70

(52) U.S. Cl. CPC G16H 50/70

(57) ABSTRACT

An estimation device includes a reception unit that receives an input of a data set for each stage, a first estimation unit that estimates a first simultaneous distribution based on a transition from a data distribution in a first stage to a data distribution in a second stage that is a stage after the first stage, a second estimation unit that estimates a second simultaneous distribution based on a transition from the estimated first simultaneous distribution to a data distribution in a third stage that is a stage after the second stage, and a calculation unit that calculates a state transition probability related to data transition from the second stage to the third stage based on the second simultaneous distribution. The estimation device can support decision making regarding future states by predicting future states through the estimation of state transitions.

This application is based upon and claims the benefit of priority from Japanese Patent Application No. 2024-144416, filed on Aug. 26, 2024 the disclosure of which is incorporated herein in its entirety by reference.

### TECHNICAL FIELD

The present disclosure relates to an estimation device and the like.

### BACKGROUND ART

In the field of healthcare and the like, there is a technology for performing future analysis of information regarding a person.

JP 2004-089267 A discloses a technique in which a probability that the same sleep state is maintained and a probability of transition from one sleep state to another sleep state are experimentally obtained in advance for each of the sleep states of the subject, and the sleep depth of the subject is estimated using the obtained probability.

### SUMMARY

An object of the present disclosure is to provide an estimation device and the like capable of estimating a state transition in consideration of a past state even in a case where there is no temporal data regarding the same target.

An estimation device according to an aspect of the present disclosure includes a reception unit that receives an input of a data set for each stage, a first estimation unit that estimates a first simultaneous distribution based on a transition from a data distribution in a first stage to a data distribution in a second stage that is a stage after the first stage, a second estimation unit that estimates a second simultaneous distribution based on a transition from the estimated first simultaneous distribution to a data distribution in a third stage that is a stage after the second stage, and a calculation unit that calculates a state transition probability related to data transition from the second stage to the third stage based on the second simultaneous distribution.

An estimation method according to an aspect of the present disclosure includes receiving an input of a data set for each stage, estimating a first simultaneous distribution based on a transition from a data distribution in a first stage to a data distribution in a second stage that is a stage after the first stage, estimating a second simultaneous distribution based on a transition from the estimated first simultaneous distribution to a data distribution in a third stage that is a stage after the second stage, and calculating a state transition probability related to data transition from the second stage to the third stage based on the second simultaneous distribution.

A non-transitory recording medium according to an aspect of the present disclosure recording a program for causing a computer to execute a process of receiving an input of a data set for each stage, a process of estimating a first simultaneous distribution based on a transition from a data distribution in a first stage to a data distribution in a second stage that is a stage after the first stage, a process of estimating a second simultaneous distribution based on a transition from the estimated first simultaneous distribution to a data distribution in a third stage that is a stage after the second stage, and a process of calculating a state transition probability related to data transition from the second stage to the third stage based on the second simultaneous distribution.

### BRIEF DESCRIPTION OF THE DRAWINGS

Exemplary features and advantages of the present invention will become apparent from the following detailed description when taken with the accompanying drawings in which:

FIG. 1 is a first block diagram illustrating an example of a functional configuration of an estimation device of the present disclosure;

FIG. 2 is a first flowchart for explaining an example of the operation of the estimation device of the present disclosure;

FIG. 3 is a second block diagram illustrating an example of a functional configuration of the estimation device of the present disclosure;

FIG. 4 is a diagram illustrating an example of a probability density distribution of the present disclosure;

FIG. 5 is a diagram for explaining an image when a transition from one probability density distribution to another probability density distribution of the present disclosure is solved as an optimal transport problem;

FIG. 6 is a second flowchart for explaining an example of the operation of the estimation device of the present disclosure;

FIG. 7 is a third block diagram illustrating an example of a functional configuration of the estimation device of the present disclosure;

FIG. 8 is a third flowchart for explaining an example of the operation of the estimation device of the present disclosure;

FIG. 9 is a fourth block diagram illustrating an example of a functional configuration of the estimation device of the present disclosure;

FIG. 10 is a fourth flowchart for explaining an example of the operation of the estimation device of the present disclosure;

FIG. 11 is a fifth flowchart for explaining an example of the operation of the estimation device of the present disclosure; and

FIG. 12 is a block diagram illustrating an example of a hardware configuration of a computer device that implements the estimation device of the present disclosure.

### EXAMPLE EMBODIMENT

Hereinafter, example embodiments of the present disclosure will be described with reference to the drawings.

### First Example Embodiment

An outline of an estimation device according to a first example embodiment will be described.

The estimation device of the present disclosure calculates a state transition probability between data using the accumulated data. In the present disclosure, an example of target data is health data that is data regarding health of a person. The health data may be, for example, a value of an inspection item in a medical examination, or may be information regarding an exercise habit of a person. The health data is not limited to this example.

The health data is accumulated in advance, for example. At this time, the accumulated health data may not be temporal data that is a result of continuous observation of a specific person for a predetermined period. The accumulated health data may be data at a predetermined time point of each of the plurality of persons. For example, the results of the medical examinations for 10,000 people in the year of t may be accumulated as health data. In the present disclosure, an example in which the estimation device estimates the data transition based on the health data will be mainly described, but the target data is not limited to this example.

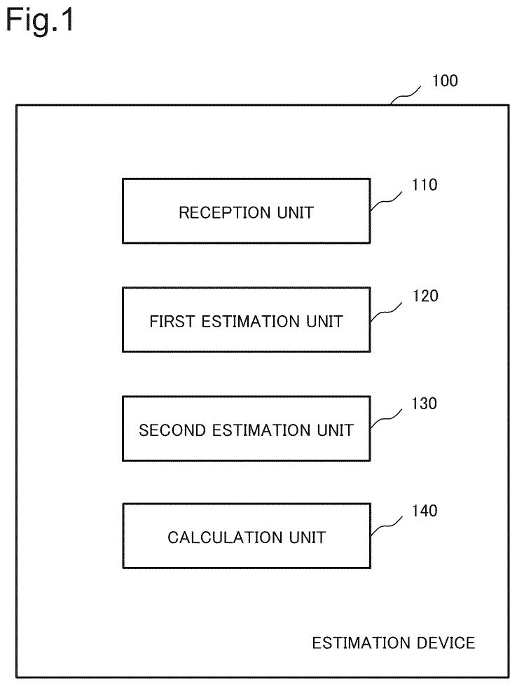

FIG. 1 is a first block diagram illustrating an example of a functional configuration of an estimation device 100. As illustrated in FIG. 1, the estimation device 100 includes a reception unit 110, a first estimation unit 120, a second estimation unit 130, and a calculation unit 140.

The reception unit 110 receives an input of a data set. The data set may be health data of a plurality of persons. For example, the data set may be a result of a medical examination performed on a plurality of persons in one year. As described above, the data set may be data regarding the result of the medical examination at a predetermined time point of each of the plurality of persons. The data set may be stored in advance in a storage device (not illustrated). The storage device may be a device included in the estimation device 100 or an external device communicably connected to the estimation device 100.

The data sets are classified by stage. The stage may be information indicating a layer when the data set is stratified. In other words, the stage can also be said to be information indicating a condition in a case where data serving as a population is classified into a subset based on a predetermined condition. For example, if the data set is health data, the data set for each stage may be health data for each age group. More specifically, in a case where the health data is data indicating blood glucose levels of a plurality of persons, the data set for each stage may include data indicating a blood glucose level of a person whose age group is 10s, data indicating a blood glucose level of a person in 20s, . . . , and data indicating a blood glucose level of a person in 80s. That is, in this example, the data set includes data indicating blood glucose levels for every age group of 10s. In this manner, the order may be determined for the stage. For example, the next stage after the stage where the age group is 10s is the stage where the age group is 20s. The age group may be any age group.

In this manner, the reception unit 110 receives the input of the data set for each stage.

The first estimation unit 120 estimates a simultaneous distribution based on data transition between predetermined stages. Specifically, the first estimation unit 120 estimates a first simultaneous distribution based on the transition from the data distribution in a first stage to the data distribution in a second stage.

It is assumed that the health data is data indicating blood glucose levels of a plurality of persons. It is assumed that the first stage is an age group of 40s and the second stage is an age group of 50s. That is, the second stage is a stage after the first stage. In this case, for example, the first estimation unit 120 estimates the transition from the distribution of the blood glucose level of a person in 40s to the distribution of the blood glucose level of a person in 50s. At this time, the first estimation unit 120 may perform estimation using an algorithm of the optimal transport problem. That is, the first estimation unit 120 estimates a likely transition from the probability distribution indicating the probability that a person in 40s having each value of the blood glucose level is present to the probability distribution indicating the probability that a person in 50s having each value of the blood glucose level is present. In the estimation, a distribution indicating a correspondence relationship between a possible value of the random variable of each probability distribution and the probability is estimated. A distribution indicating this correspondence relationship is referred to as a simultaneous distribution. The simultaneous distribution estimated by the first estimation unit 120 is referred to as a first simultaneous distribution.

In this manner, the first estimation unit 120 estimates the first simultaneous distribution based on the transition from the data distribution in the first stage to the data distribution in the second stage which is the stage after the first stage.

The second estimation unit 130 estimates the simultaneous distribution based on the transition from the estimated first simultaneous distribution to the data distribution in a third stage. The third stage is a stage subsequent to the second stage. For example, when the second stage is in the age group of 50s and the third stage is in the age group of 60s, the third stage is a stage after the second stage.

It is assumed that the first simultaneous distribution is a simultaneous distribution based on the transition from the distribution of the blood glucose level of a person in 40s to the distribution of the blood glucose level of a person in 50s as described above. It is assumed that the third stage is in the age group of 60s. At this time, for example, the second estimation unit 130 estimates the transition from the first simultaneous distribution to the distribution of the blood glucose level of the person in 60s. At this time, the second estimation unit 130 may perform estimation using an algorithm of the optimal transport problem, similarly to the first estimation unit 120.

That is, the second estimation unit 130 estimates the simultaneous distribution indicating the correspondence relationship between the possible values of the random variable and the probability in the first simultaneous distribution and the probability distribution indicating the probability that a person in 60s having each value of the blood glucose level is present. The simultaneous distribution estimated by the second estimation unit 130 is referred to as a second simultaneous distribution. It can be said that the second simultaneous distribution indicates a transition from the distribution of the blood glucose level of the person in 50s to the distribution of the blood glucose level of the person in 60s in consideration of the distribution of the blood glucose level of the person in 40s.

In this manner, the second estimation unit 130 estimates the second simultaneous distribution based on the transition from the estimated first simultaneous distribution to the data distribution in the third stage, which is the stage after the second stage.

Then, the calculation unit 140 calculates the state transition probability regarding the data transition from the second stage to the third stage based on the second simultaneous distribution.

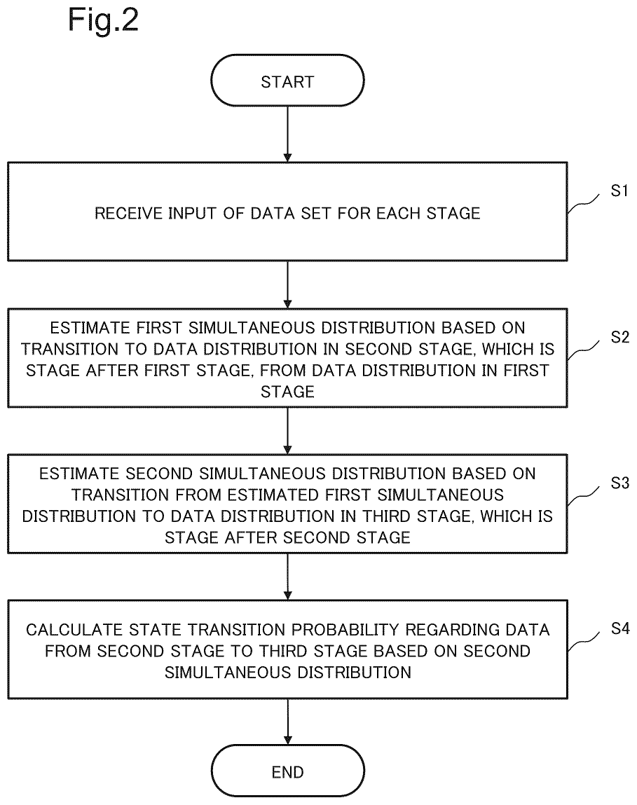

Next, an exemplary operation of the estimation device 100 will be described with reference to FIG. 2. In the present disclosure, each step of the flowchart is represented using a number assigned to each step, such as “S1”.

FIG. 2 is a flowchart for explaining an exemplary operation of the estimation device 100.

The reception unit 110 receives an input of a data set for each stage (S1).

The first estimation unit 120 estimates the first simultaneous distribution based on a transition from the data distribution in the first stage to a data distribution in the second stage which is a stage after the first stage (S2).

The second estimation unit 130 estimates the second simultaneous distribution based on the transition from the estimated first simultaneous distribution to the data distribution in the third stage which is the stage after the second stage (S3).

The calculation unit 140 calculates the state transition probability regarding the data transition from the second stage to the third stage based on the second simultaneous distribution (S4).

As described above, the estimation device 100 of the first example embodiment receives the input of the data set for each stage. The estimation device 100 estimates the first simultaneous distribution based on a transition from the data distribution in the first stage to a data distribution in the second stage which is a stage after the first stage. Further, the estimation device 100 estimates the second simultaneous distribution based on the transition from the estimated first simultaneous distribution to the data distribution in the third stage, which is the stage after the second stage. Then, the estimation device 100 calculates the state transition probability regarding the data transition from the second stage to the third stage based on the second simultaneous distribution.

Therefore, the estimation device can support decision making regarding future states by predicting future states through the estimation of state transitions.

The estimation device 100 estimates the transition to the data distribution in the third stage using the first simultaneous distribution based on the transition from the data distribution in the first stage to the data distribution in the second stage. As a result, the estimation device 100 can consider the data in the first stage when estimating the transition from the data distribution in the second stage to the data distribution in the third stage. The estimation device 100 estimates the first simultaneous distribution. That is, the estimation device 100 does not perform a method that requires temporal data of the same person, such as experimentally obtaining a probability related to a transition. That is, even in a case where there is no temporal data regarding the same person, the estimation device 100 can estimate the state transition in consideration of the past state.

### Second Example Embodiment

Next, an estimation device according to a second example embodiment will be described. In the second example embodiment, a further example of the estimation device described in the first example embodiment will be described. Also in the second example embodiment, an example in which the estimation device 100 estimates the data transition based on the health data will be mainly described, but the target data is not limited to this example. Part of the description of content overlapping with that of the first example embodiment will be omitted.

### <Details of Estimation Device 100>

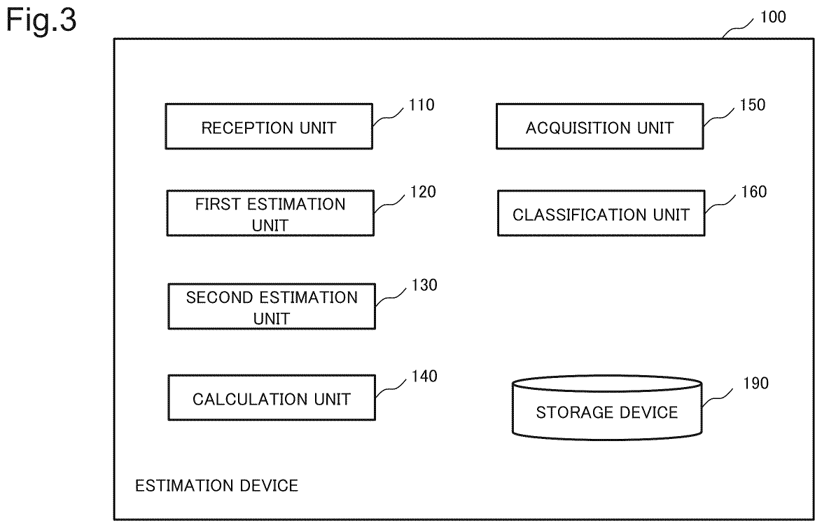

FIG. 3 is a block diagram illustrating an exemplary functional configuration of an estimation device 100. The estimation device 100 includes a reception unit 110, a first estimation unit 120, a second estimation unit 130, and a calculation unit 140. The estimation device 100 may include an acquisition unit 150 and a classification unit 160. Further, the estimation device 100 may include a storage device 190. The storage device 190 may be a device included in the estimation device 100 or an external device communicably connected to the estimation device 100.

The estimation device 100 is, for example, a device provided in a terminal device such as a personal computer. The terminal device is a device operated by a user. The estimation device 100 is not limited to this example, and may be a device implemented in a server device communicably connected to a terminal device via a wired or wireless network. The estimation device 100 may perform various types of processing in accordance with an instruction from the terminal device.

The estimation device 100 may be communicably connected to a further device via a wired or wireless network. For example, the estimation device 100 may be communicable with an external server device having health data. The external server device is, for example, a device managed by a hospital, a local government, a company, or the like.

The reception unit 110 receives an input of a data set for each stage. At this time, the data set is stored in the storage device 190. For example, the reception unit 110 may receive reading of a data set stored in the storage device 190 as an input of the data set according to an instruction from the terminal device.

The storage device 190 stores health data. The reception unit 110 may receive the health data as a data set. The health data is acquired by the acquisition unit 150.

The acquisition unit 150 acquires health data. Specifically, the acquisition unit 150 acquires the health data from an external server device that manages the health data. For example, it is assumed that the results of the medical examination for 10,000 people in the year of t are managed by an external server device. The acquisition unit 150 acquires the results of the medical examination for 10,000 people in the year of t as health data from an external server device. The health data may be information relevant to an inspection item of a medical examination. The health data is a result of a medical examination that each of the 10,000 persons received at one time point in the year of t. As described above, the data set may be data measured at a time point of each of a plurality of targets instead of data indicating a temporal change of the same target. The acquisition unit 150 stores the acquired health data in the storage device 190.

The health data acquisition method is not limited to this example. For example, there may be a recording medium storing health data. At this time, the terminal device reads the health data from the recording medium. Then, the acquisition unit 150 may acquire the health data read by the terminal device.

In this manner, the acquisition unit 150 acquires health data that is data regarding health of each of a plurality of persons at a predetermined time point.

The health data is processed by the classification unit 160, for example. Then, the processed health data may be stored in the storage device 190. The classification unit 160 processes the data acquired by the acquisition unit 150 into a data set according to the condition. For example, the classification unit 160 classifies the health data for each age group. For example, the classification unit 160 classifies health data for each age group of 10s. The present invention is not limited to this example, and the classification unit 160 may classify the health data at an arbitrary age interval. For example, the classification unit 160 may classify the health data for every 1 year old.

At this time, the classification unit 160 may extract specific data from the health data and classify the extracted data for each age group. For example, it is assumed that the health data includes information indicating height, weight, blood pressure, blood glucose level, HbA1c, and Body Math Index (BMI). At this time, the classification unit 160 may classify data indicating the blood glucose level and BMI among the health data for each age group.

The condition to be classified and the data to be extracted may be information according to an instruction from the terminal device. That is, a user who operates the terminal device inputs information indicating a condition to be classified and data to be extracted to the terminal device. The terminal device transmits the input information to the estimation device 100. The classification unit 160 processes the health data using the information indicating the classification condition and the data to be extracted transmitted from the terminal device.

The classification unit 160 generates a distribution related to the acquired data. Specifically, the classification unit 160 generates a probability density distribution for each condition based on the acquired data. For example, the classification unit 160 generates a distribution obtained by plotting data indicating the blood glucose level and BMI for each age group. The distribution generated at this time is a two-dimensional distribution relating to blood glucose level and BMI. Then, the classification unit 160 generates a probability density distribution indicating an existence probability of each value of the blood glucose level and the BMI. When a data value of one dimension is xᵢ and a data value of another dimension is xⱼ, the probability density distribution can be expressed as p([xᵢ, xⱼ]). In this manner, the classification unit 160 generates a probability density distribution for each age group regarding the acquired health data.

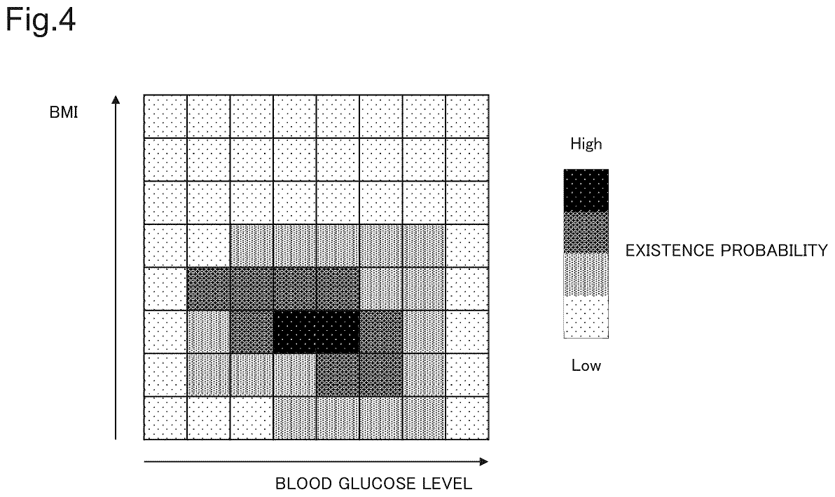

At this time, the classification unit 160 may classify data in each distribution into a data group. In this case, the probability density distribution is a distribution indicating the existence probability for each data group. FIG. 4 is a diagram illustrating an example of a probability density distribution. The probability density distribution illustrated in FIG. 4 is based on each of the blood glucose level and BMI. In the example of FIG. 4, 64 cells are illustrated. This cell is a data group in which data indicating the blood glucose level and BMI of each person is classified. Then, the existence probability for each data group is illustrated. In this manner, the classification unit 160 may classify data in each distribution of health data for each age group into a data group. The classification unit 160 may generate a two-dimensional or more distribution. For example, the classification unit 160 may generate a probability density distribution around each of the blood glucose level, BMI, and the average number of steps per day. The probability density distribution divided by the cells as illustrated in FIG. 4 is relevant to a peripheral distribution based on each value of the health data. The probability density distribution to be treated hereinafter may be a probability distribution as illustrated in FIG. 4 or a probability distribution that does not take the form of a peripheral distribution.

The reception unit 110 receives the health data classified for each condition as a data set for each stage. For example, the reception unit 110 may receive the probability density distribution related to the health data for each age group as described above as a data set for each stage.

The first estimation unit 120 estimates a first simultaneous distribution between the stages. Specifically, the first estimation unit 120 estimates the first simultaneous distribution based on a transition from the distribution of health data in a first age group to the distribution of health data in a second age group. Here, the second age group is an age group after the first age group. Hereinafter, the distribution of health data in the first age group is referred to as a first distribution. The distribution of the health data in the second age group is referred to as a second distribution.

The first estimation unit 120 estimates the transition from the first distribution to the second distribution using an algorithm of an optimal transport problem (hereinafter, referred to as an optimal transport algorithm). The optimal transport algorithm is an algorithm for obtaining a transport method that optimizes a cost necessary for transitioning a predetermined probability distribution to another probability distribution.

Specifically, with respect to distributions μ and v in a probability space X, the fact that a distribution π in a direct product X² is coupling means that the following Expressions 1 and 2 hold.

$$π ( \cdot \times x ) = μ \qquad [ Math . 1 ]$$

$$π ( X \times \cdot ) = v \qquad [ Math . 2 ]$$

The entire coupling is defined as Π(μ, v). A cost function for transporting an element x included in the distribution u to an element y included in the distribution v is c(x, y). In this case, for example, in the following Expression 3, the coupling that minimizes the cost is referred to as optimal transport. The distribution x at this time is relevant to the simultaneous distribution.

$${\int }_{{X}^{2}} c ( x , y ) π ( dxdy ) , π ∈ Π ( μ , v ) \qquad [ Math . 3 ]$$

That is, it is possible to calculate a set of data before transport and data of a transport destination, which optimizes the cost of transport from the data distribution in the first stage to the data distribution in the second stage, by the optimal transport algorithm.

The first estimation unit 120 estimates the first simultaneous distribution based on a transition from the first distribution that is a distribution of health data in the first age group to the second distribution that is a distribution of health data in the second age group that is an age group after the first age group. At this time, the first estimation unit 120 solves the transition from the first distribution to the second distribution as the optimal transport problem. Here, the first distribution and the second distribution are probability density distributions. That is, the first estimation unit 120 solves the transition from the probability density distribution related to the health data in the first age group to the probability density distribution related to the health data in the second age group as the optimal transport problem.

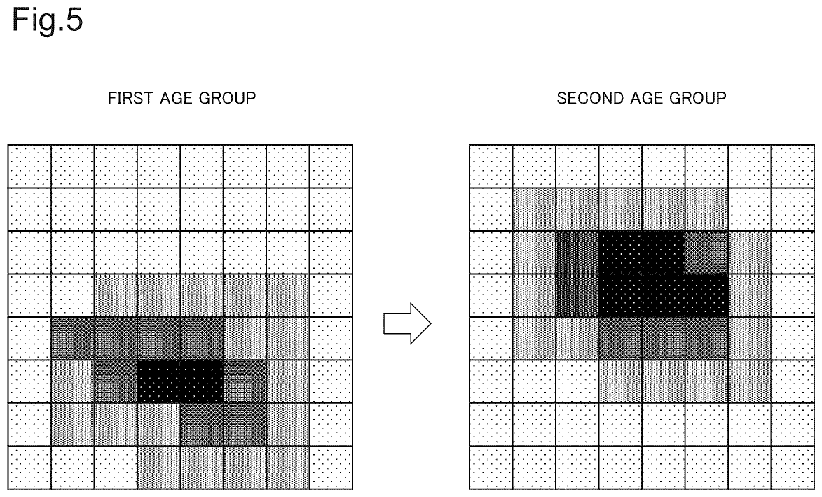

FIG. 5 is a diagram for explaining an image when a transition from one probability density distribution to another probability density distribution is solved as an optimal transport problem. FIG. 5 illustrates a probability density distribution related to health data in the first age group and a probability density distribution related to health data in the second age group. Solving, by the first estimation unit 120, the transition from the first distribution to the second distribution as the optimal transport problem is relevant to estimating to which cell in the probability density distribution of the second age group the probability of transition of each cell in the probability density distribution of the first age group is higher. That is, the first estimation unit 120 estimates the first simultaneous distribution based on the transition from each data group in the first distribution to each data group in the second distribution.

For example, μ is a probability density distribution related to health data in the first age group, and v is a probability density distribution related to health data in the second age group. At this time, the first estimation unit 120 estimates the first simultaneous distribution π∈Π(μ, v) using Expression 3.

The second estimation unit 130 estimates the second simultaneous distribution based on a transition from the estimated first simultaneous distribution to a distribution of health data in a third age group that is an age group after the second age group. Hereinafter, the distribution of health data in the third age group is also referred to as a third distribution.

Similarly to the first estimation unit 120, the second estimation unit 130 estimates the transition from the first simultaneous distribution to the data distribution in the third stage using the optimal transport algorithm. That is, the second estimation unit 130 estimates the second simultaneous distribution by using an optimal transport algorithm that optimizes the cost of transport from the first simultaneous distribution to the data distribution in the third stage and calculates a set of data before transport and data of the transport destination.

For example, the second estimation unit 130 solves the transition from the first simultaneous distribution to the third distribution as the optimal transport problem. The third distribution is a probability density distribution. At this time, a new cost function is defined. ξ is a third distribution (that is, probability density distribution regarding health data in the third age group). An element of the distribution ξ is z. At this time, c((x, y), z) is defined as a new cost function. The entire coupling of the distribution p in the direct product X²×X is defined as Π(π, ξ). Then, the second estimation unit 130 estimates the second simultaneous distribution ρ∈Π(π, ξ) based on Expression 3 in which the new cost function is defined.

The calculation unit 140 estimates the state transition probability related to the transition of the health data based on the simultaneous distribution. In the optimal transport problem, obtaining an optimal coupling (simultaneous distribution) is equivalent to obtaining an optimal state transition. That is, the state transition probability can be obtained from the simultaneous distribution. For example, the calculation unit 140 calculates a state transition probability ρ(z|x, y) from the second age group to the third age group based on second simultaneous distribution ρ.

As described above, the state transition probability ρ(z|x, y) is information based on the result of solving the transition from the first simultaneous distribution to the third distribution as the optimal transport problem. Therefore, in the state transition probability ρ(z|x, y), health data in the first age group is also considered. That is, in the calculation of the state transition probability ρ(z|x, y), Markov property is not assumed for the state transition. That is, the calculation unit 140 calculates the non-Markov state transition probability ρ(z|x, y) from the second age group to the third age group in consideration of the health data in the first age group. In this example, the example of solving the optimal transport problem using the probability density distribution (peripheral distribution) in which the first distribution and the second distribution are divided into cells as illustrated in FIG. 4 has been described. The example of solving the optimal transport problem is not limited to this example. For example, the first distribution and the second distribution may be distributions in which the health data is not classified into a data group, and each value of the health data and the existence probability are indicated.

As described above, the calculation unit 140 can calculate the state transition probability from the data distribution in the second stage to the data distribution in the third stage based on the second simultaneous distribution.

Similarly, the calculation unit 140 can calculate the state transition probability π (y|x) from the first age group to the second age group based on first simultaneous distribution π. That is, the calculation unit 140 can calculate the state transition probability from the data distribution in the first stage to the data distribution in the second stage based on the first simultaneous distribution. The calculation unit 140 may store the calculated state transition probability in the storage device 190.

### <Operation Example of Estimation Device 100>

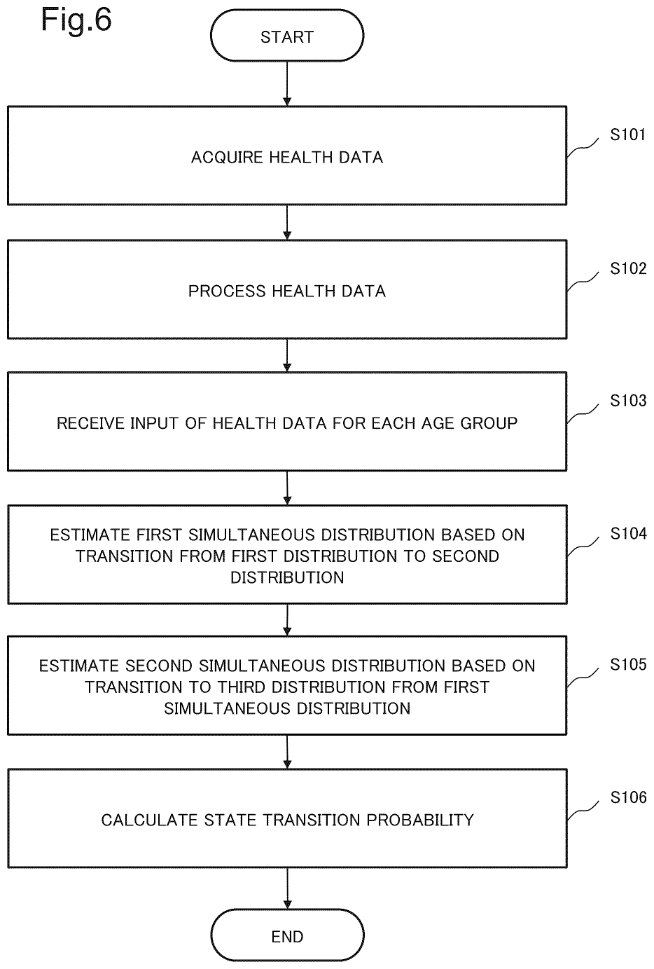

Next, an exemplary operation of the estimation device 100 will be described with reference to FIG. 6.

FIG. 6 is a second flowchart for explaining an exemplary operation of the estimation device 100. Specifically, FIG. 6 is a flowchart for explaining an exemplary operation when the estimation device 100 calculates the state transition probability between predetermined stages. In the present operation example, it is assumed that data indicating the blood glucose level and BMI is acquired as health data. The present operation example shows an example in which the estimation device 100 calculates a state transition probability from the distribution of health data in the age group of 50s to the distribution of health data in the age group of 60s.

The acquisition unit 150 acquires health data (S101). For example, the acquisition unit 150 acquires health data from an external server device. Then, the acquisition unit 150 stores the health data in the storage device 190. The classification unit 160 processes the health data (S102). For example, the classification unit 160 classifies the health data for each age group. Then, the classification unit 160 generates a probability density distribution for each age group of 10s regarding the health data. At this time, the probability density distribution is a distribution indicating the existence probability of each value of the blood glucose level and BMI.

The reception unit 110 receives an input of health data for each age group (S103). For example, the reception unit 110 receives reading of the health data stored in the storage device 190 as an input of the health data.

The first estimation unit 120 estimates a first simultaneous distribution based on a transition from the distribution (first distribution) of health data in the first age group to the distribution (second distribution) of health data in the second age group (S104). Here, the first age group is in 40s. The second age group is in 50s. That is, the first estimation unit 120 estimates the first simultaneous distribution based on the transition from the probability density distribution related to the health data of 40s to the probability density distribution related to the health data of 50s.

The second estimation unit 130 estimates the second simultaneous distribution based on the transition from the first simultaneous distribution to the distribution (third distribution) of health data in the third age group (S105). Here, the third age group is in 60s. That is, the second estimation unit 130 estimates the second simultaneous distribution based on the transition from the simultaneous distribution considering the transition of the health data from 40s to 50s to the probability density distribution related to the health data of 60s.

Then, the calculation unit 140 estimates the state transition probability based on the estimated second simultaneous distribution (S106). Specifically, the calculation unit 140 calculates, based on the second simultaneous distribution, a state transition probability from a probability density distribution related to health data of 50s to a probability density distribution related to health data of 60s.

The present operation example is merely an example. That is, the operation of the estimation device 100 of the present disclosure is not limited to this example. The estimation device 100 may not necessarily estimate the state transition probability in consecutive stages. In the present operation example, an example of obtaining a state transition probability from health data of 50s to health data of 60s in consideration of health data of 40s based on health data in which age groups are classified for every 10s is illustrated. Not limited to this example, for example, the estimation device 100 may obtain the state transition probability from the health data of 50s to the health data of 70s in consideration of the health data of 40s. The estimation device 100 may obtain the state transition probability from the health data of 50s to the health data of 60s in consideration of the health data of 30s.

As described above, the estimation device 100 of the second example embodiment receives the input of the data set for each stage. The estimation device 100 estimates the first simultaneous distribution based on a transition from the data distribution in the first stage to a data distribution in the second stage which is a stage after the first stage. Further, the estimation device 100 estimates the second simultaneous distribution based on the transition from the estimated first simultaneous distribution to the data distribution in the third stage, which is the stage after the second stage. Then, the estimation device 100 calculates the state transition probability regarding the data transition from the second stage to the third stage based on the second simultaneous distribution.

Specifically, for example, the estimation device 100 receives health data, which is data regarding health at a predetermined time point of each of a plurality of persons for each age group, as a data set for each stage. The estimation device 100 estimates the first simultaneous distribution based on a transition from the first distribution that is a distribution of health data in the first age group to the second distribution that is a distribution of health data in the second age group that is an age group after the first age group. Furthermore, the estimation device 100 estimates the second simultaneous distribution based on a transition from the estimated first simultaneous distribution to a third distribution that is a distribution of health data in a third age group that is an age group after the second age group. Then, the estimation device 100 calculates a state transition probability related to the transition of the health data from the second age group to the third age group based on the second simultaneous distribution.

In this manner, the estimation device 100 estimates the transition from the distribution of the health data in the first age group to the distribution of the health data in the third age group by using the first simultaneous distribution based on the transition to the distribution of the health data in the second age group. As a result, the estimation device 100 can consider the health data in the first age group when estimating the transition from the distribution of the health data in the second age group to the distribution of the health data in the third age group. The estimation device 100 estimates the first simultaneous distribution. That is, the estimation device 100 does not perform a method that requires temporal data of the same person, such as experimentally obtaining a probability related to a transition. That is, even in a case where there is no temporal data of the same person, the estimation device 100 can estimate the state transition in consideration of the past state.

Furthermore, by calculating the state transition probability in this manner, for example, it is possible to predict what value the health data will have in a case where the predetermined person has reached the age of the third age group based on the health data of the predetermined person with the age relevant to the second age group.

The estimation device 100 may estimate the first simultaneous distribution using an optimal transport algorithm that optimizes the cost of transport from the data distribution in the first stage to the data distribution in the second stage and calculates a set of data before transport and data of a transport destination. Further, the estimation device 100 may estimate the second simultaneous distribution using an optimal transport algorithm that optimizes the cost of transport from the first simultaneous distribution to the data distribution at the third stage and calculates a set of data before transport and data of a transport destination.

As a result, the estimation device 100 can calculate the state transition probability from the optimal transport between the distributions even if the target of the data in each stage is not the same. That is, the estimation device 100 can estimate the state transition even in a case where there is no temporal data of the same target.

### Third Example Embodiment

Next, an estimation device according to a third example embodiment will be described. In the third example embodiment, an example of predicting the health state of the target person based on the calculated state transition probability will be described. Also in the third example embodiment, an example in which the estimation device estimates the data transition based on the health data will be mainly described, but the target data is not limited to this example. The description of part of the content overlapping with the content of the first example embodiment and the second example embodiment will be omitted.

### <Details of Estimation Device 101>

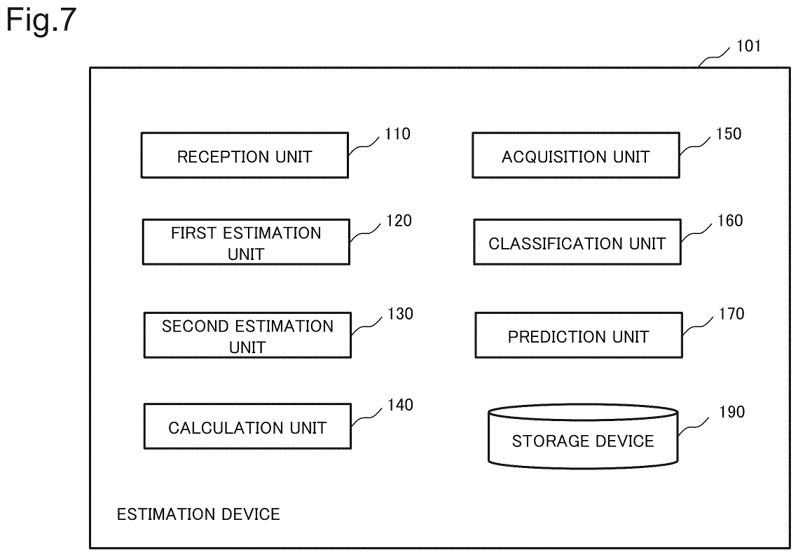

An estimation device 101 is a device in which a further functional unit is added to the estimation device 100. FIG. 7 is a block diagram illustrating an exemplary functional configuration of the estimation device 101. The estimation device 101 includes a reception unit 110, a first estimation unit 120, a second estimation unit 130, a calculation unit 140, an acquisition unit 150, and a classification unit 160. The estimation device 101 may include a prediction unit 170. Further, the estimation device 101 may include a storage device 190.

Similarly to the estimation device 100, the estimation device 101 may be a device provided in a terminal device, or may be a device implemented in a server device communicably connected to the terminal device via a wired or wireless network.

The estimation device 101 predicts future health data for the target person using the state transition probability calculated in advance. In the present example embodiment, it is assumed that the first age group is 40s, the second age group is 50s, and the third age group is 60s. Then, it is assumed that a state transition probability from health data in 50s to health data in 60s is calculated.

The acquisition unit 150 acquires health data of the target person. For example, it is assumed that the state transition probability is calculated from the probability density distribution regarding the blood glucose level and BMI. At this time, the acquisition unit 150 acquires health data indicating the blood glucose level and BMI of the target person.

The prediction unit 170 predicts the transition of the health data of the target person. In other words, the prediction unit 170 predicts the value of the future health data of the target person based on the health data of the target person. Specifically, the prediction unit 170 predicts the value of the health data in a case where the target person has reached the age relevant to the third age group. At this time, the second age group is an age group relevant to the age of the target person.

For example, it is assumed that the target person is 51 years old. In this case, the age of the target person is relevant to the second age group. The prediction unit 170 specifies which data group the health data of the target person is classified into in the probability density distribution related to the health data in 50s. Then, the prediction unit 170 predicts to which data group the specified data group transitions in the probability density distribution related to the health data in 60s based on the state transition probability.

In this manner, the prediction unit 170 predicts a data group in the third distribution, which is a transition destination based on the state transition probability of the data group in the second distribution relevant to the health data of the target person, as the health data in a case where the target person reaches the age of the third age group.

### <Operation Example of Estimation Device 101>

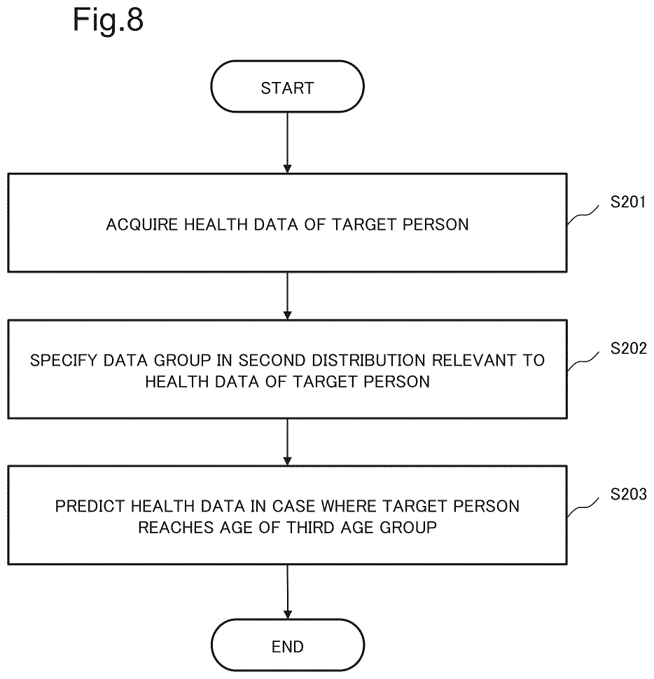

Next, an exemplary operation of the estimation device 101 will be described with reference to FIG. 8.

FIG. 8 is a third flowchart for explaining an exemplary operation of the estimation device 101. Specifically, FIG. 8 is a flowchart illustrating an exemplary operation when the estimation device 101 predicts the health state of the target person.

The acquisition unit 150 acquires health data of the target person (S201). For example, the acquisition unit 150 acquires the health data of the target person from the terminal device.

The prediction unit 170 specifies a data group in the second distribution relevant to the health data of the target person (S202). For example, the prediction unit 170 specifies which data group the health data of the target person is classified into in the probability density distribution related to the health data in the second age group.

Then, the prediction unit 170 predicts the health data in a case where the target person reaches the age of the third age group based on the state transition probability (S203). Specifically, the prediction unit 170 predicts a data group in the probability density distribution regarding the health data in the third age group, which is the transition destination based on the state transition probability of the specified data group, as the health data in a case where the target person reaches the age of the third age group.

The present operation example is merely an example. That is, the operation of the estimation device 101 of the present disclosure is not limited to this example.

As described above, the estimation device 101 of the third example embodiment acquires the health data of the target person. At this time, the second age group is an age group relevant to the age of the target person. Then, the estimation device 101 predicts a data group in the third distribution, which is a transition destination based on the state transition probability of the data group in the second distribution relevant to the health data of the target person, as the health data in a case where the target person reaches the age of the third age group. Thus, the estimation device 101 can predict the future health state of the target person.

### Fourth Example Embodiment

Next, an estimation device according to a fourth example embodiment will be described. In the fourth example embodiment, a further example of predicting the health state of the target person based on the calculated state transition probability will be described. Also in the fourth example embodiment, an example in which the estimation device estimates the data transition based on the health data will be mainly described, but the target data is not limited to this example. The description of part of the content overlapping with the content of the first example embodiment, the second example embodiment, and the third example embodiment will be omitted.

### <Details of Estimation Device 102>

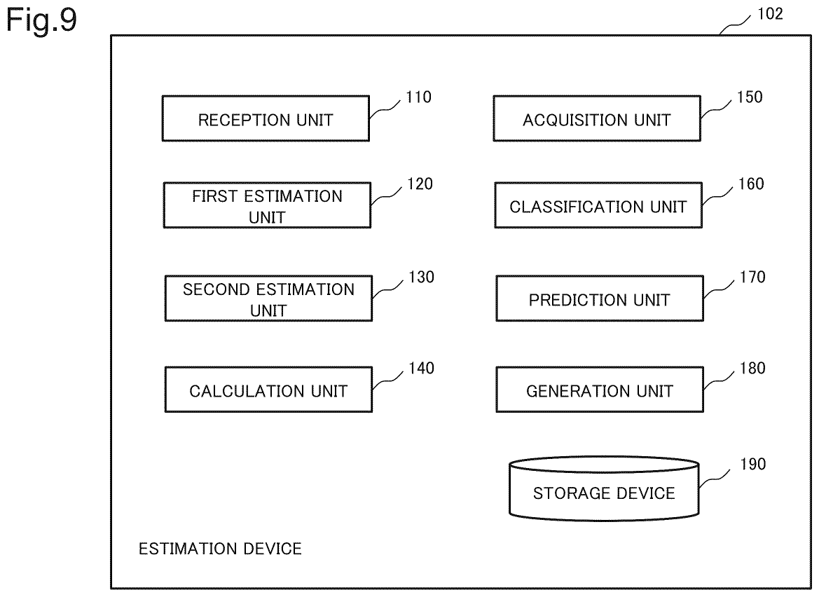

An estimation device 102 is a device in which a further functional unit is added to the estimation device 101. FIG. 9 is a block diagram illustrating an exemplary functional configuration of the estimation device 102. The estimation device 102 includes a reception unit 110, a first estimation unit 120, a second estimation unit 130, a calculation unit 140, an acquisition unit 150, a classification unit 160, and a prediction unit 170. The estimation device 101 may include a generation unit 180. Further, the estimation device 102 may include a storage device 190.

Similarly to the estimation device 101, the estimation device 102 may be a device provided in a terminal device, or may be a device implemented in a server device communicably connected to the terminal device via a wired or wireless network.

The estimation device 102 calculates a state transition probability between stages in advance. Then, the estimation device 102 generates a learning model using the calculated state transition probability. The estimation device 102 predicts future health data for the target person using the generated learning model. In the present example embodiment, a stage of generating the learning model is referred to as a generation phase. A stage of performing prediction is referred to as a prediction phase.

### (Generation Phase)

It is assumed that a data set for each stage is stored in the storage device 190 in advance. For example, it is assumed that a probability density distribution related to health data for each age group is stored in the storage device 190.

The reception unit 110 receives an input of a probability density distribution related to health data for each age group. In the present example embodiment, the probability density distribution is a distribution related to health data for every 10s. It is assumed that there are eight types of probability density distributions from 10s to 80s.

The first estimation unit 120 estimates a first simultaneous distribution regarding adjacent stages. Specifically, the first estimation unit 120 estimates the first simultaneous distribution based on the transition of the data distribution from the first stage to the stage adjacent to the first stage in the data set for each stage. For example, the first estimation unit 120 estimates a first simultaneous distribution based on transition from a probability density distribution related to health data of 10s to a probability density distribution related to health data of 20s. The first estimation unit 120 may estimate the first simultaneous distribution based on the transition from the probability density distribution related to the health data of 20s to the probability density distribution related to the health data of 30s. Similarly, the first estimation unit 120 may estimate the first simultaneous distribution based on the transition of the data distribution for each set of adjacent stages based on the probability density distribution regarding the health data between the adjacent stages. Hereinafter, the first simultaneous distribution (N<M) based on the transition of the probability density distribution related to the health data in the age group from the Ns to the Ms is referred to as a first simultaneous distribution between Ns and Ms.

The second estimation unit 130 estimates the second simultaneous distribution based on the transition from the first simultaneous distribution to the distribution of health data in another age group. For example, the second estimation unit 130 estimates the second simultaneous distribution based on the transition from the first simultaneous distribution between 10s and 20s to the distribution of health data in the age group in 30s.

Further, the second estimation unit 130 estimates a further simultaneous distribution using the estimated second simultaneous distribution. Specifically, the estimated second simultaneous distribution is considered as a first simultaneous distribution. Then, the second estimation unit 130 estimates the second simultaneous distribution based on the transition from the simultaneous distribution regarded as the first simultaneous distribution to the data distribution in the further adjacent stage. For example, it is assumed that the second estimation unit 130 estimates the second simultaneous distribution based on the transition from the first simultaneous distribution between 10s and 20s to the distribution of health data in the age group in 30s. The second simultaneous distribution estimated at this time is regarded as a first simultaneous distribution between 10s and 30s. Therefore, the second estimation unit 130 estimates the second simultaneous distribution based on the transition from the first simultaneous distribution between 10s and 30s to the distribution of health data in the age group of 40s. Similarly, since the second simultaneous distribution is regarded as the first simultaneous distribution between 10s and 40s, the second estimation unit 130 estimates the second simultaneous distribution based on the transition from the first simultaneous distribution between 10s and 40s to the distribution of health data in the age group of 50s. The second estimation unit 130 continues the processing until the second simultaneous distribution based on the transition to the distribution of health data in the age group of 80s is estimated. That is, the second estimation unit 130 continues the processing until estimating the second simultaneous distribution based on the transition to the data distribution at the last stage in the data set.

In this manner, the first estimation unit 120 first estimates the first simultaneous distribution based on the transition of the data distribution between the adjacent stages. The second estimation unit 130 estimates the second simultaneous distribution based on the transition from the first simultaneous distribution to the data distribution in an adjacent stage after the adjacent stage. Then, the second estimation unit 130 regards the estimated second simultaneous distribution as a first simultaneous distribution, and repeats a process of estimating a second simultaneous distribution based on a transition to a data distribution in an adjacent stage until the adjacent stage is the last stage.

The calculation unit 140 calculates a state transition probability related to data transition between the stages. At this time, the calculation unit 140 calculates the state transition probability regarding the data transition between the stages based on each of the first simultaneous distribution and the second distribution.

The generation unit 180 generates a machine learning model. Specifically, the generation unit 180 generates a prediction model that outputs data in another stage, which is a transition destination of data in one stage, based on the calculated state transition probability. The prediction model is relevant to a machine learning model that learns a relationship between a data distribution in one stage and a data distribution in another stage. Alternatively, the generation unit 180 may generate a prediction model that outputs a data group of data in another stage, which is a transition destination of a data group of a data distribution in one stage, based on the calculated state transition probability. The generated prediction model is a machine learning model that receives age and health data as inputs, and outputs health data in which the input health data transitions after a predetermined period or a data group thereof.

In this manner, the generation unit 180 generates the machine learning model in which the relationship between the data in the stage before transition and the data in the stage after transition is learned based on the state transition probability.

### (Prediction Phase)

The acquisition unit 150 acquires health data of the target person.

The prediction unit 170 estimates the future health state of the target person using the machine learning model. Specifically, the prediction unit 170 inputs the health data of the target person and the age of the target person to the machine learning model. The health data in the age group equal to or higher than the age of the target person is output by the machine learning model. That is, the health data in a case where the target person reaches the age after the lapse of the predetermined period is output. The prediction unit 170 predicts the health data as future health data of the target person.

For example, it is assumed that the target person is 51 years old. Then, it is assumed that health data in a case where the target person is in 70s is predicted. In this case, the prediction unit 170 inputs, for example, information indicating that the age is 51 years old and the health data of the target person to the machine learning model. At this time, the machine learning model outputs the health data in the age group of 70s, which is the transition destination of the input health data. The prediction unit 170 outputs the output health data as health data in a case where the target person is in 70s.

The machine learning model may be a model that outputs health data after a lapse of a specific period with respect to the input health data. For example, the machine learning model may output health data in a case where the target person reaches an age group after 20 years. The machine learning model may be a model that outputs a transition of health data until after a lapse of a specific period. For example, the transition of the health data until the target person reaches the age group after 20 years may be output.

### <Operation Example of Estimation Device 102>

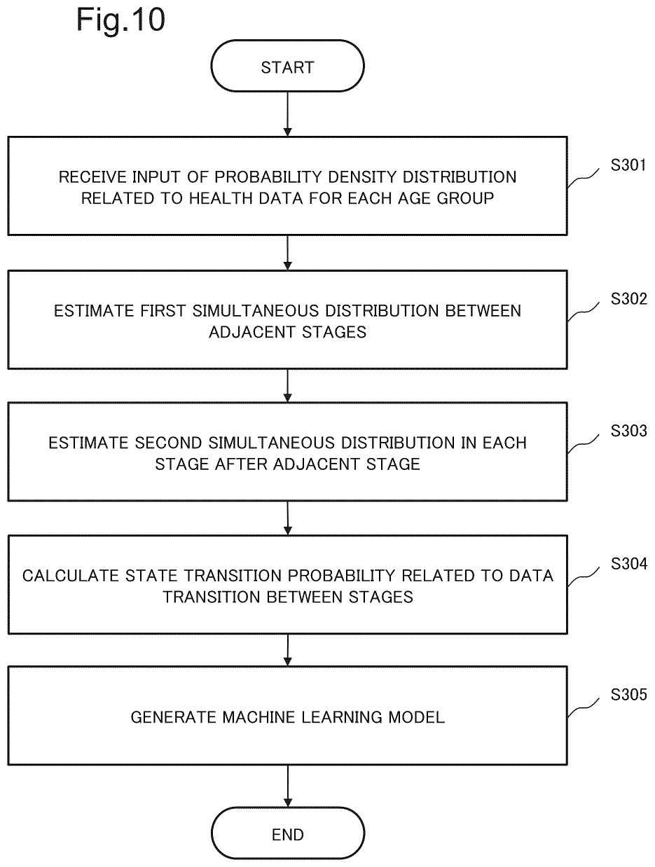

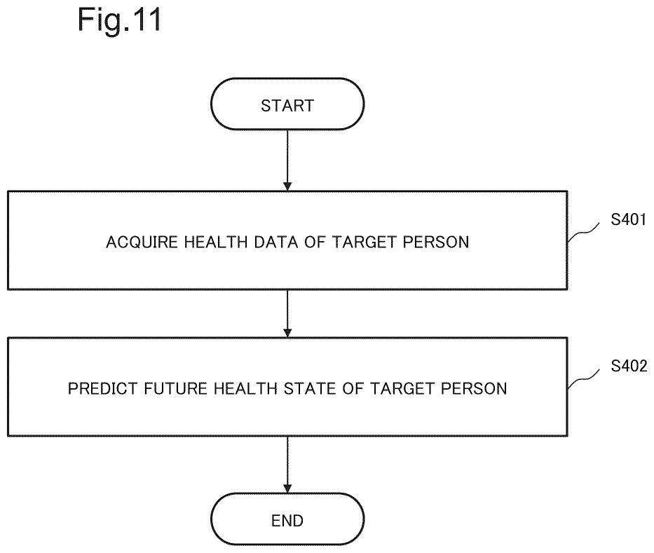

Next, an example of the operation of the estimation device 102 will be described with reference to FIGS. 10 and 11.

FIG. 10 is a fourth flowchart for explaining an exemplary operation of the estimation device 102. Specifically, FIG. 10 is a flowchart for explaining an example of the operation of the estimation device 102 in the generation phase. In the operation example of FIG. 10, it is assumed that a probability density distribution related to health data for each age group is stored in the storage device 190 in advance.

The reception unit 110 receives an input of a probability density distribution related to health data for each age group (S301). For example, the reception unit 110 receives an input of a probability density distribution related to health data for every 10s.

The first estimation unit 120 estimates the first simultaneous distribution between the adjacent stages (S302). For example, the first estimation unit 120 estimates the first simultaneous distribution based on the transition of the data distribution between the adjacent stages based on the probability density distribution regarding the health data between the age group of 10s that is the first stage and the age group of 20s that is the adjacent stage.

The second estimation unit 130 estimates the second simultaneous distribution in each stage after the adjacent stage (S303). Specifically, the second estimation unit 130 estimates each of the second simultaneous distributions based on the transition from the first simultaneous distribution to the data distribution in an adjacent stage after the adjacent stage. Then, the second estimation unit 130 regards the estimated second simultaneous distribution as a first simultaneous distribution, and repeats a process of estimating a second simultaneous distribution based on a transition to a data distribution in an adjacent stage until the adjacent stage is the last stage.

The calculation unit 140 calculates a state transition probability related to data transition between the stages (S304). Then, the generation unit 180 generates a machine learning model based on the calculated state transition probability (S305). Specifically, the generation unit 180 generates the machine learning model in which the relationship between the data in the stage before transition and the data in the stage after transition is learned based on the state transition probability.

FIG. 11 is a fifth flowchart for explaining an exemplary operation of the estimation device 102. Specifically, FIG. 11 is a flowchart for explaining an example of the operation of the estimation device 102 in the prediction phase.

The acquisition unit 150 acquires health data of the target person (S401). For example, the acquisition unit 150 acquires the health data of the target person from the terminal device.

The prediction unit 170 estimates the future health state of the target person using the machine learning model (S402). Specifically, the prediction unit 170 inputs information indicating the age of the target person and the health data to the machine learning model. Then, the health data is output by the machine learning model. The prediction unit 170 outputs the output health data as health data in a case where the target person reaches an age after a predetermined period has elapsed.

The present operation example is merely an example. That is, the operation of the estimation device 102 of the present disclosure is not limited to this example.

As described above, the estimation device 102 of the fourth example embodiment estimates the first simultaneous distribution based on the transition of the data distribution between the adjacent stages. The estimation device 102 estimates the second simultaneous distribution based on the transition from the first simultaneous distribution to the data distribution in an adjacent stage after the adjacent stage. The estimation device 102 regards the estimated second simultaneous distribution as a first simultaneous distribution, and repeats a process of estimating a second simultaneous distribution based on a transition to a data distribution in an adjacent stage until the adjacent stage is the last stage. Further, the estimation device 102 calculates a state transition probability related to the data transition between the stages based on each of the second simultaneous distribution. Then, the estimation device 102 generates the machine learning model in which the relationship between the data in the stage before transition and the data in the stage after transition is learned based on the state transition probability.

### <Modifications>

In the present disclosure, the example in which the estimation device estimates the data transition based on the health data has been mainly described. That is, an example in which the estimation device is used in the healthcare or medical field has been mainly described. However, the example to which the estimation device is applied is not limited thereto. For example, the estimation device may also be applied to a case of estimating state transitions of various machines.

For example, in a case where the measurement data measured for the operating state of the machine is acquired, the estimation device may receive, as the data set for each stage, the measurement data of each state based on the secular change from the state in which the machine normally operates to the state in which the machine fails. The estimation device may estimate a first simultaneous distribution based on the distribution of the measurement data between the states, and estimate a second simultaneous distribution based on a transition from the first simultaneous distribution to the data distribution in another state. Then, the estimation device may calculate the state transition probability related to the transition between the states.

### <Exemplary Hardware Configuration of Estimation Device>

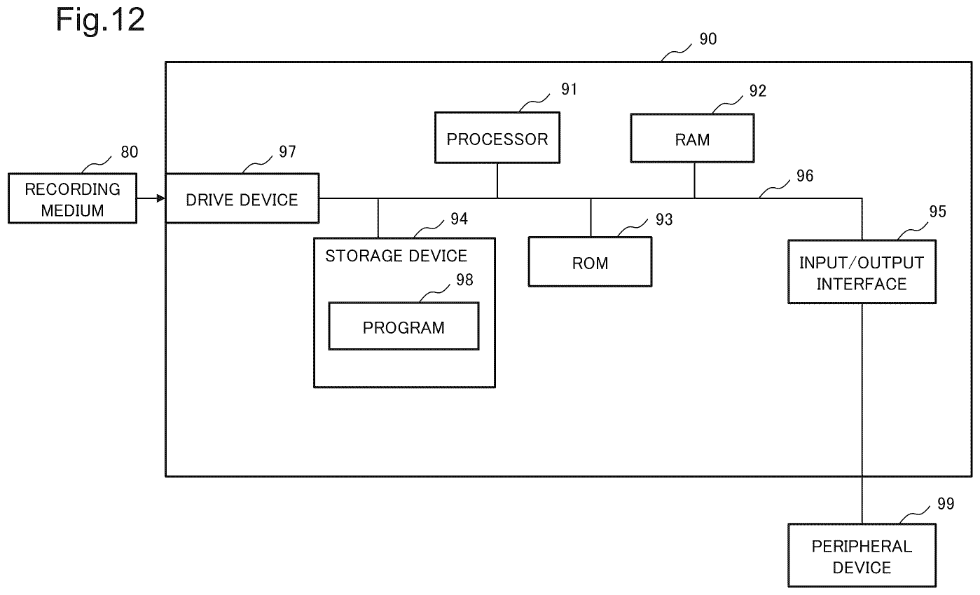

Hardware constituting the estimation devices of the first, second, third, and fourth example embodiments will be described. FIG. 12 is a block diagram illustrating an example of a hardware configuration of a computer device constituting the estimation device according to each example embodiment. In a computer device 90, the estimation device and the estimation method described in each example embodiment and each modification are achieved. For example, the estimation device and the like described in each example embodiment and each modification may have the hardware configuration illustrated in FIG. 12.

As illustrated in FIG. 12, the computer device 90 includes a processor 91, a random access memory (RAM) 92, a read only memory (ROM) 93, a storage device 94, an input/output interface 95, a bus 96, and a drive device 97. The estimation device and the like may be achieved by a plurality of electric circuits.

The storage device 94 stores a program (computer program) 98. The processor 91 executes the program 98 of the present estimation device using the RAM 92. Specifically, for example, the program 98 includes a program that causes a computer to execute the processing illustrated in FIG. 2, FIG. 6, FIG. 8, FIG. 10, and FIG. 11. When the processor 91 executes the program 98, the function of each configuration of the present estimation device is implemented. The program 98 may be stored in the ROM 93. The program 98 may be recorded in a recording medium 80 and read using a drive device 97, or may be transmitted from an external device (not illustrated) to the computer device 90 via a network (not illustrated).

The input/output interface 95 exchanges data with a peripheral device (keyboard, mouse, display device, etc.) 99. The input/output interface 95 functions as a means for acquiring or outputting data. The bus 96 connects the components.

There are various modifications of the method of achieving the estimation device. For example, each configuration included in the estimation device can be achieved as a dedicated device. The estimation device can be achieved based on a combination of a plurality of devices.

A processing method of causing a recording medium to record a program for achieving each configuration in the functions of each example embodiment, reading the program recorded in the recording medium as a code, and a computer executing the program are also included in the scope of each example embodiment. That is, a computer-readable recording medium is included in the scope of each example embodiment. A recording medium recording the above-described program and the program itself are also included in each example embodiment.

The recording medium is, for example, a floppy (registered trademark) disk, a hard disk, an optical disk, a magneto-optical disk, a compact disc (CD)-ROM, a magnetic tape, a nonvolatile memory card, or a ROM, but is not limited to this example. The program recorded in the recording medium is not limited to a program that executes processing by itself, and programs that operate on an operating system (OS) to execute processing in cooperation with other software and functions of an extension board are also included in the scope of each example embodiment.

In JP 2004-089267 A, information on a target person is predicted by estimating a state of a transition destination from a current state of the target person based on a probability related to a state transition. Here, for example, in a case where data regarding health is predicted, it may be required to consider not only the current state but also the past state. That is, there is a case where it is required to consider the past state in estimating the state transition.

In JP 2004-089267 A, when a state transition related to a person is estimated, a probability related to the transition is calculated based on data accumulated in advance. For calculation of such a probability, for example, temporal data in which the same person is observed for a predetermined period is used. On the other hand, it may be difficult to accumulate temporal data regarding the same subject. In a case where there is no temporal data regarding the same subject, it is difficult to experimentally obtain the probability regarding the transition.

An object of the present disclosure is to provide an estimation device and the like capable of estimating a state transition in consideration of a past state even in a case where there is no temporal data regarding the same target.

According to the present disclosure, even in a case where there is no temporal data regarding the same person, it is possible to estimate a state transition in consideration of a past state.

Some or all of the above example embodiments may be described as the following Supplementary Notes, but are not limited to the following.

### <Supplementary Notes>

### [Supplementary Note 1]

An estimation device including:
a reception unit that receives an input of a data set for each stage;
a first estimation unit that estimates a first simultaneous distribution based on a transition from a data distribution in a first stage to a data distribution in a second stage that is a stage after the first stage;
a second estimation unit that estimates a second simultaneous distribution based on a transition from the estimated first simultaneous distribution to a data distribution in a third stage that is a stage after the second stage; and
a calculation unit that calculates a state transition probability related to data transition from the second stage to the third stage based on the second simultaneous distribution.

### [Supplementary Note 2]

The estimation device according to Supplementary Note 1, in which
the reception unit receives health data, which is data regarding health at a predetermined time point of each of a plurality of persons for each age group, as a data set for each stage,
the first estimation unit estimates the first simultaneous distribution based on a transition from a first distribution that is a distribution of the health data in a first age group to a second distribution that is a distribution of the health data in a second age group that is an age group after the first age group,
the second estimation unit estimates the second simultaneous distribution based on a transition from the estimated first simultaneous distribution to a third distribution that is a distribution of the health data in a third age group that is an age group after the second age group, and
the calculation unit calculates a state transition probability related to a transition of the health data from the second age group to the third age group based on the second simultaneous distribution.

### [Supplementary Note 3]

The estimation device according to Supplementary Note 2, including:
an acquisition unit that acquires the health data, the health data being data regarding health of each of a plurality of persons at a predetermined time point; and
a classification unit that classifies data in each distribution of the health data for each age group into a data group, in which
the first estimation unit estimates the first simultaneous distribution based on a transition from each data group in the first distribution to each data group in the second distribution, and
the second estimation unit estimates the second simultaneous distribution based on a transition from each data group in the first simultaneous distribution to each data group in the third distribution.

### [Supplementary Note 4]

The estimation device according to Supplementary Note 3, including a prediction unit that predicts a transition of health data of a target person based on the state transition probability, in which
the acquisition unit acquires health data of the target person,
the second age group is an age group relevant to an age of the target person, and
the prediction unit predicts a data group in the third distribution, which is a transition destination based on the state transition probability of a data group in the second distribution, relevant to the health data of the target person, as health data in a case where the target person reaches an age of the third age group.

### [Supplementary Note 5]

The estimation device according to Supplementary Note 3, in which
the classification unit generates a probability density distribution for each age group regarding the acquired health data,
each of the first distribution, the second distribution, and the third distribution is the probability density distribution, and
the probability density distribution indicates an existence probability for each of the data groups.

### [Supplementary Note 6]

The estimation device according to Supplementary Note 1, in which
the first estimation unit estimates the first simultaneous distribution by using an optimal transport algorithm that optimizes a cost of transport from a data distribution in the first stage to a data distribution in the second stage and calculates a set of data before transport and data of a transport destination, and
the second estimation unit estimates the second simultaneous distribution using an optimal transport algorithm that optimizes a cost of transport from the first simultaneous distribution to the data distribution in the third stage and calculates a set of data before transport and data of a transport destination.

### [Supplementary Note 7]

The estimation device according to Supplementary Note 1, in which
the data set is data regarding a result of a medical examination at a predetermined time point of each of a plurality of persons.

### [Supplementary Note 8]

The estimation device according to Supplementary Note 1, further including a generation unit that generates a machine learning model, in which
the first estimation unit estimates the first simultaneous distribution based on a transition of a data distribution between adjacent stages,
the second estimation unit estimates the second simultaneous distribution based on a transition from the first simultaneous distribution to a data distribution in an adjacent stage after the adjacent stage,
the second estimation unit regards the estimated second simultaneous distribution as a first simultaneous distribution, and repeats a process of estimating the second simultaneous distribution based on a transition to a data distribution in an adjacent stage until an adjacent stage is the last stage,
the calculation unit calculates a state transition probability related to a data transition between stages based on each of the second simultaneous distributions, and
the generation unit generates a machine learning model in which a relationship between data in a stage before transition and data in a stage after transition is learned based on a state transition probability.

### [Supplementary Note 9]

An estimation method including:
receiving an input of a data set for each stage;
estimating a first simultaneous distribution based on a transition from a data distribution in a first stage to a data distribution in a second stage that is a stage after the first stage;
estimating a second simultaneous distribution based on a transition from the estimated first simultaneous distribution to a data distribution in a third stage that is a stage after the second stage; and
calculating a state transition probability related to data transition from the second stage to the third stage based on the second simultaneous distribution.

### [Supplementary Note 10]

A non-transitory recording medium recording a program for causing a computer to execute:
a process of receiving an input of a data set for each stage;
a process of estimating a first simultaneous distribution based on a transition from a data distribution in a first stage to a data distribution in a second stage that is a stage after the first stage;
a process of estimating a second simultaneous distribution based on a transition from the estimated first simultaneous distribution to a data distribution in a third stage that is a stage after the second stage; and
a process of calculating a state transition probability related to data transition from the second stage to the third stage based on the second simultaneous distribution.

Some or all of the configurations described in Supplementary Notes 2 to 8 dependent on the above-described Supplementary Note 1 can also be dependent on Supplementary Notes 9 and 10 by the same dependency relationship as in Supplementary Notes 2 to 8. Some or all of the configurations described as a Supplementary Note can be similarly dependent on various recording means or systems for recording various hardware, software, and software without departing from the above-described example embodiments.

The previous description of embodiments is provided to enable a person skilled in the art to make and use the present disclosure. Moreover, various modifications to these example embodiments will be readily apparent to those skilled in the art, and the generic principles and specific examples defined herein may be applied to other embodiments without the use of inventive faculty. Therefore, the present disclosure is not intended to be limited to the example embodiments described herein but is to be accorded the widest scope as defined by the limitations of the claims and equivalents.

Further, it is noted that the inventor's intent is to retain all equivalents of the claimed invention even if the claims are amended during prosecution.

1. An estimation device comprising:
at least one memory storing instructions; and
at least one processor configured to access the at least one memory and execute the instructions to:
receive an input of a data set for each stage;
estimate a first simultaneous distribution based on a transition from a data distribution in a first stage to a data distribution in a second stage that is a stage after the first stage;
estimate a second simultaneous distribution based on a transition from the estimated first simultaneous distribution to a data distribution in a third stage that is a stage after the second stage; and
calculate a state transition probability related to data transition from the second stage to the third stage based on the second simultaneous distribution.

2. The estimation device according to claim 1, wherein
the at least one processor is further configured to execute the instructions to:
receive health data, which is data regarding health at a predetermined time point of each of a plurality of persons for each age group, as a data set for each stage,
estimate the first simultaneous distribution based on a transition from a first distribution that is a distribution of the health data in a first age group to a second distribution that is a distribution of the health data in a second age group that is an age group after the first age group;
estimate the second simultaneous distribution based on a transition from the estimated first simultaneous distribution to a third distribution that is a distribution of the health data in a third age group that is an age group after the second age group; and
calculate a state transition probability related to a transition of the health data from the second age group to the third age group based on the second simultaneous distribution.

3. The estimation device according to claim 2, wherein
the at least one processor is further configured to execute the instructions to:
acquire the health data, the health data being data regarding health of each of a plurality of persons at a predetermined time point;
classify data in each distribution of the health data for each age group into a data group;
estimate the first simultaneous distribution based on a transition from each data group in the first distribution to each data group in the second distribution; and
estimate the second simultaneous distribution based on a transition from each data group in the first simultaneous distribution to each data group in the third distribution.

4. The estimation device according to claim 3, wherein
the at least one processor is further configured to execute the instructions to:
acquire health data of the target person; and
predict a data group in the third distribution, which is a transition destination based on the state transition probability of a data group in the second distribution, relevant to the health data of the target person, as health data in a case where the target person reaches an age of the third age group, wherein
the second age group is an age group relevant to an age of the target person.

5. The estimation device according to claim 3, wherein
the at least one processor is further configured to execute the instructions to:
generate a probability density distribution for each age group regarding the acquired health data, wherein
each of the first distribution, the second distribution, and the third distribution is the probability density distribution, and
the probability density distribution indicates an existence probability for each of the data groups.

6. The estimation device according to claim 1, wherein
the at least one processor is further configured to execute the instructions to:
estimate the first simultaneous distribution by using an optimal transport algorithm that optimizes a cost of transport from a data distribution in the first stage to a data distribution in the second stage and calculates a set of data before transport and data of a transport destination; and
estimate the second simultaneous distribution using an optimal transport algorithm that optimizes a cost of transport from the first simultaneous distribution to the data distribution in the third stage and calculates a set of data before transport and data of a transport destination.

7. The estimation device according to claim 1, wherein
the data set is data regarding a result of a medical examination at a predetermined time point of each of a plurality of persons.

8. The estimation device according to claim 1, wherein
the at least one processor is further configured to execute the instructions to:
estimate the first simultaneous distribution based on a transition of a data distribution between adjacent stages;
estimate the second simultaneous distribution based on a transition from the first simultaneous distribution to a data distribution in an adjacent stage after the adjacent stage;
repeat a process of estimating the second simultaneous distribution based on a transition to a data distribution in an adjacent stage until an adjacent stage is the last stage, by regarding the estimated second simultaneous distribution as a first simultaneous distribution, and;
calculate a state transition probability related to a data transition between stages based on each of the second simultaneous distribution; and
generate a machine learning model in which a relationship between data in a stage before transition and data in a stage after transition is learned based on a state transition probability.

9. An estimation method comprising:
receiving an input of a data set for each stage;
estimating a first simultaneous distribution based on a transition from a data distribution in a first stage to a data distribution in a second stage that is a stage after the first stage;
estimating a second simultaneous distribution based on a transition from the estimated first simultaneous distribution to a data distribution in a third stage that is a stage after the second stage; and
calculating a state transition probability related to data transition from the second stage to the third stage based on the second simultaneous distribution.

10. A non-transitory recording medium recording a program for causing a computer to execute:
a process of receiving an input of a data set for each stage;
a process of estimating a first simultaneous distribution based on a transition from a data distribution in a first stage to a data distribution in a second stage that is a stage after the first stage;
a process of estimating a second simultaneous distribution based on a transition from the estimated first simultaneous distribution to a data distribution in a third stage that is a stage after the second stage; and
a process of calculating a state transition probability related to data transition from the second stage to the third stage based on the second simultaneous distribution.
